(19) United States
(12) Patent Application Publication
(10) Pub. No.: US 2024/0029842 A1
(43) Pub. Date: Jan. 25, 2024

(54) INFORMATION PROCESSING APPARATUS, INFORMATION PROCESSING SYSTEM, INFORMATION PROCESSING METHOD, AND INFORMATION PROCESSING PROGRAM

(71) Applicant: FUJIFILM CORPORATION, Tokyo (JP)

(72) Inventors: Yasuhisa KANEKO, Kanagawa (JP); Tomohide HIRAGAMI, Kanagawa (JP)

(21) Appl. No.: 18/481,200

(22) Filed: Oct. 4, 2023

Related U.S. Application Data
(63) Continuation of application No. PCT/JP2022/016910, filed on Mar. 31, 2022.

(30) Foreign Application Priority Data
Apr. 15, 2021 (JP) 2021-069310

(51) Int. Cl. G16H 10/40

(52) U.S. Cl. CPC G16H 10/40

(57) ABSTRACT

An information processing apparatus comprising at least one processor, wherein the processor is configured to: acquire first biological information over time related to each of a plurality of subjects; for each subject, derive a timing suitable for measuring second biological information of the subject different from the first biological information based on the first biological information; and schedule a period for measuring the second biological information for each subject based on the derived timing.

### CROSS REFERENCE TO RELATED APPLICATIONS

This application is a continuation of International Application No. PCT/JP2022/016910, filed on Mar. 31, 2022, which claims priority from Japanese Patent Application No. 2021-069310, filed on Apr. 15, 2021. The entire disclosure of each of the above applications is incorporated herein by reference.

### BACKGROUND

### Technical Field

The present disclosure relates to an information processing apparatus, an information processing system, an information processing method, and an information processing program.

### Related Art

In the related art, a technology for monitoring certain biological information and setting a condition related to measurement of another biological information based on a monitoring result has been known. For example, JP2009-172397A discloses setting an imaging condition of an X-ray computed tomography imaging apparatus in the case of imaging a subject based on a heart rate measured from the identical subject.

Some illnesses may have a change in appearance of a lesion depending on a state (for example, after eating, after exercise, and after wake-up) of the subject. For example, diabetic retinopathy that is a complication of diabetes is diagnosed based on the appearance of the lesion such as capillary hemangioma and retinal hemorrhage in a fundus image obtained by imaging a fundus. These lesions related to the diabetic retinopathy may be more noticeable in a case where a blood glucose level of the subject is high. That is, in a case where the fundus image can be captured at a timing when the blood glucose level of the subject is high, it is possible to appropriately diagnose the diabetic retinopathy and contribute to its early detection.

In recent years, a technology that provides ability to measure the biological information for appropriate diagnosis by presenting an appropriate timing of measurement with respect to the biological information in which the appearance of the lesion changes depending on the state of the subject, as described above, has been desired.

### SUMMARY

The present disclosure provides an information processing apparatus, an information processing system, an information processing method, and an information processing program that can measure biological information for appropriate diagnosis.

A first aspect of the present disclosure is an information processing apparatus comprising at least one processor, in which the processor is configured to acquire first biological information over time related to each of a plurality of subjects, for each subject, derive a timing suitable for measuring second biological information of the subject different from the first biological information based on the first biological information, and schedule a period for measuring the second biological information for each subject based on the derived timing.

A second aspect of the present disclosure is provided such that in the first aspect, the processor may be configured to perform scheduling such that the periods for each subject do not overlap with each other.

A third aspect of the present disclosure is provided such that in the first or second aspect, the processor may be configured to, for each subject, derive the timing based on a change in time of the first biological information.

A fourth aspect of the present disclosure is provided such that in any one of the first to third aspects, the processor may be configured to, for each subject, derive a start timing and an end timing of a period suitable for measuring the second biological information based on the first biological information.

A fifth aspect of the present disclosure is provided such that in the fourth aspect, the processor may be configured to, in a case where the timings derived for each subject overlaps with each other, schedule the period for measuring the second biological information by prioritizing a subject of which at least one of the start timing or the end timing is earlier.

A sixth aspect of the present disclosure is provided such that in the fourth or fifth aspect, the processor may be configured to, in a case where the timings derived for each subject overlaps with each other, schedule the period for measuring the second biological information by placing a subject of which a period from the start timing to the end timing is longer in a middle in order.

A seventh aspect of the present disclosure is provided such that in any one of the first to sixth aspects, the processor may be configured to, for each subject, derive a most suitable timing for measuring the second biological information from the subject based on the first biological information.

An eighth aspect of the present disclosure is provided such that in any one of the first to seventh aspects, the processor may be configured to, for each subject, predict a change in time of the first biological information, and derive the timing based on the predicted first biological information.

A ninth aspect of the present disclosure is provided such that in the eighth aspect, the processor may be configured to predict the change in time of the first biological information based on past data related to the first biological information for each subject.

A tenth aspect of the present disclosure is provided such that in the eighth or ninth aspect, the processor may be configured to monitor progress of the first biological information for each subject after scheduling the period, and in a case where a difference between the progress of the first biological information and the predicted change in time of the first biological information falls outside an allowable range, predict the change in time of the first biological information again, derive the timing again, and schedule the period again.

An eleventh aspect of the present disclosure is provided such that in any one of the first to tenth aspects, the processor may be configured to present the scheduled period.

A twelfth aspect of the present disclosure is provided such that in any one of the first to eleventh aspects, the processor may be configured to instruct a second measurement apparatus that measures the second biological information to measure the second biological information in the scheduled period.

A thirteenth aspect of the present disclosure is provided such that in any one of the first to twelfth aspects, the first biological information may aperiodically change depending on behavior of the subject.

A fourteenth aspect of the present disclosure is provided such that in any one of the first to thirteenth aspects, the first biological information may indicate at least one of a body temperature, a heart rate, an electrocardiogram, an electromyogram, a blood pressure, an arterial oxygen saturation, a blood glucose level, or a lipid level, and the second biological information may indicate the electrocardiogram, an electroencephalogram, a medical image captured by a medical image capturing apparatus, and a result of at least one of a hematological test, an infectious disease test, a biochemical test, or a urine test.

A fifteenth aspect of the present disclosure is provided such that the information processing apparatus according to any one of the first to fourteenth aspects may further comprise a first measurement apparatus that measures the first biological information, and a second measurement apparatus that measures the second biological information.

A sixteenth aspect of the present disclosure is an information processing system comprising the information processing apparatus according to any one of the first to fourteenth aspects, a first measurement apparatus that measures the first biological information, and a second measurement apparatus that measures the second biological information.

A seventeenth aspect of the present disclosure is an information processing system comprising the information processing apparatus according to any one of the first to fourteenth aspects, and a first measurement apparatus that measures the first biological information, in which the information processing apparatus further includes a second measurement apparatus that measures the second biological information.

An eighteenth aspect of the present disclosure is an information processing system comprising the information processing apparatus according to any one of the first to fourteenth aspects, and a second measurement apparatus that measures the second biological information, in which the information processing apparatus further includes a first measurement apparatus that measures the first biological information.

A nineteenth aspect of the present disclosure is an information processing method comprising, via a computer, acquiring first biological information of a subject over time, and deriving a timing suitable for measuring second biological information of the subject different from the first biological information based on the first biological information.

A twentieth aspect of the present disclosure is an information processing program causing a computer to execute a process comprising acquiring first biological information of a subject over time, and deriving a timing suitable for measuring second biological information of the subject different from the first biological information based on the first biological information.

According to the aspects, the information processing apparatus, the information processing system, the information processing method, and the information processing program of the present disclosure can measure biological information for appropriate diagnosis.

### BRIEF DESCRIPTION OF THE DRAWINGS

FIG. 1 is a schematic configuration diagram of an information processing system.

FIG. 2 is an example of first biological information and second biological information.

FIG. 3 is a block diagram illustrating an example of a hardware configuration of an information processing apparatus.

FIG. 4 is a block diagram illustrating an example of a functional configuration of the information processing apparatus.

FIG. 5 is a diagram for describing timing derivation processing based on the first biological information.

FIG. 6 is a diagram for describing the timing derivation processing based on the first biological information.

FIG. 7 is a diagram illustrating an example of guidance corresponding to each timing.

FIG. 8 is a diagram for describing processing of detecting a maximum value of the first biological information.

FIG. 9 is an example of a screen on which each derived timing is presented.

FIG. 10 is a flowchart illustrating an example of first information processing.

FIG. 11 is a flowchart illustrating an example of the timing derivation processing.

FIG. 12 is an example of a screen on which a derived schedule is presented.

FIG. 13 is an example of a screen on which a schedule derived again is presented.

FIG. 14 is a flowchart illustrating an example of second information processing.

FIG. 15 is a schematic configuration diagram illustrating a modification example of the information processing system.

FIG. 16 is a schematic configuration diagram illustrating a modification example of the information processing system.

### DETAILED DESCRIPTION

Hereinafter, embodiments according to the disclosed technology will be described in detail with reference to the drawings.

### First Exemplary Embodiment

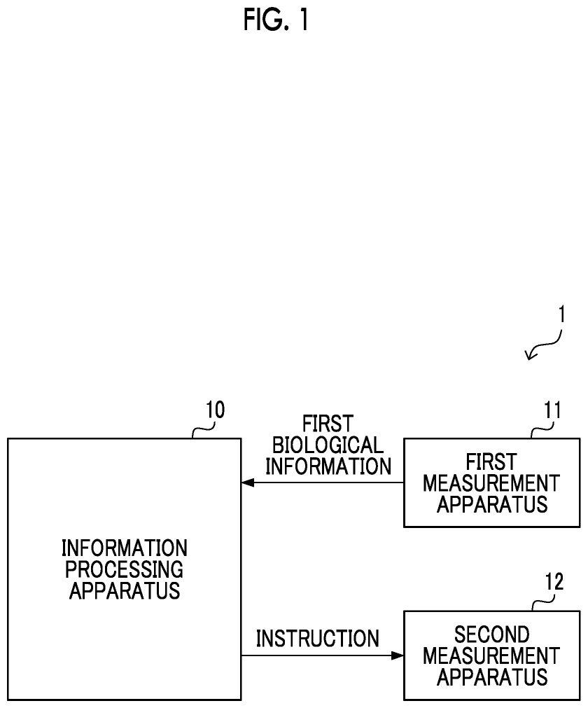

An example of a configuration of an information processing system 1 according to the first exemplary embodiment will be described with reference to FIG. 1. As illustrated in FIG. 1, the information processing system 1 comprises an information processing apparatus 10, at least one first measurement apparatus 11, and at least one second measurement apparatus 12. The information processing apparatus 10 and the first measurement apparatus 11, and the information processing apparatus 10 and the second measurement apparatus 12 can communicate with each other through wired or wireless communication.

The first measurement apparatus 11 has a function of measuring first biological information of a user over time. The first biological information may be information indicating at least one of, for example, a body temperature, a heart rate, an electrocardiogram, an electromyogram, a blood pressure, an arterial oxygen saturation (SpO2), a blood glucose level, or a lipid level. In this case, for example, a wearable terminal such as a smartwatch comprising a thermometer, a heart rate monitor, a self-monitoring blood glucose meter, and a sensor that measures biological information such as the heart rate and the arterial oxygen saturation can be applied as the first measurement apparatus 11.

The first biological information may aperiodically change depending on behavior of a subject. The behavior of the subject includes, for example, eating, exercise, and sleeping. For example, the blood glucose level as an example of the first biological information is known to rise after the subject eats. In addition, for example, the body temperature as an example of the first biological information is known to rise after the subject exercises.

The second measurement apparatus 12 has a function of measuring second biological information of the user in a one-time manner. The second biological information is a different type of biological information from the first biological information. The second biological information may be information indicating at least one of, for example, the electrocardiogram, an electroencephalogram, a medical image captured by a medical image capturing apparatus, or a result of at least one of a hematological test, an infectious disease test, a biochemical test, or a urine test. The medical image capturing apparatus is an apparatus that performs, for example, computed radiography (CR), computed tomography (CT), magnetic resonance imaging (MRI), ultrasound image diagnosis, fundus imaging, positron emission tomography (PET), and photoacoustic imaging (PAI). The medical image as the second biological information can be obtained using these medical image capturing apparatuses as the second measurement apparatus 12.

The hematological test is a test for obtaining, for example, a leukocyte count, an erythrocyte count, and a hemoglobin concentration as a test result. The biochemical test is a test for obtaining various indicators related to, for example, enzymes, proteins, glucose, lipids, and electrolytes as a test result. The infectious disease test is a test for obtaining presence or absence of infection caused by various infectious diseases such as, for example, influenza infection and novel coronavirus infection as a test result. The urine test is a test for obtaining, for example, glucose in urine, protein in urine, and occult blood in urine as a test result. In the case of using these various test results as the second biological information, a known analysis apparatus that analyzes, for example, blood and urine as a specimen can be applied as the second measurement apparatus 12.

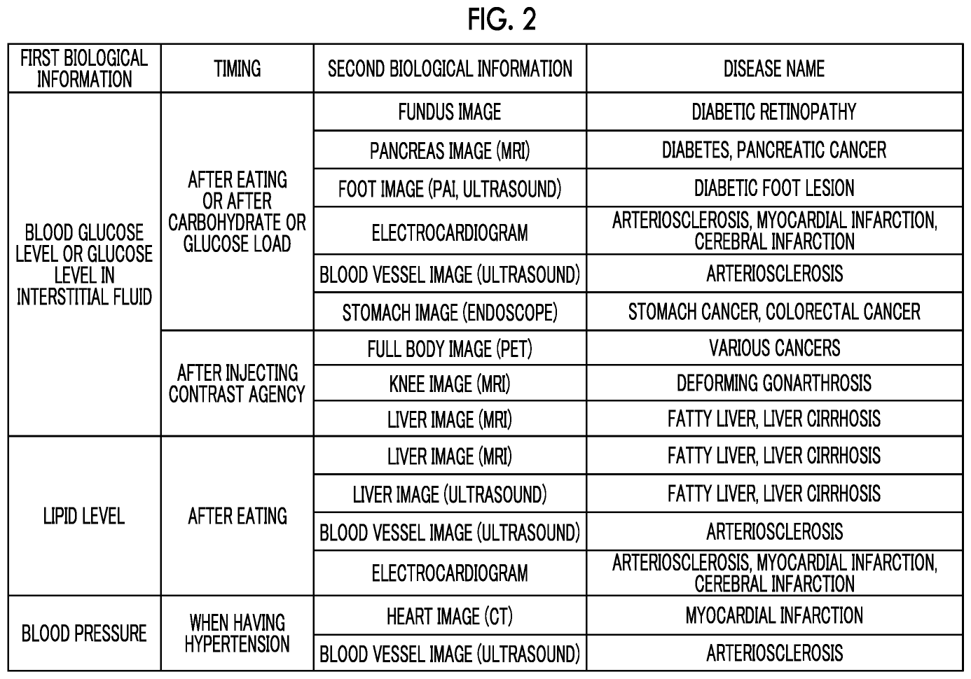

In the present exemplary embodiment, the first biological information and the second biological information are pieces of biological information that are known to be correlated with each other in advance. FIG. 2 illustrates an example of a set of the first biological information and the second biological information correlated with each other. In addition, FIG. 2 illustrates a “disease name” diagnosed based on the second biological information. As described above, since the second biological information is biological information measured in a one-time manner, a state suitable for diagnosis is required at a timing of measuring the second biological information. Whether or not the second biological information is in the state suitable for diagnosis can be estimated by monitoring the first biological information. For example, in order to diagnose the diabetic retinopathy, it is preferable to capture a fundus image as the second biological information at a timing after eating, specifically, near a peak of a high blood glucose spike after eating. The peak of the high blood glucose spike after eating can be estimated by monitoring the blood glucose level as the first biological information.

Therefore, as illustrated in FIG. 1, the first measurement apparatus 11 transmits the first biological information of the subject measured over time to the information processing apparatus 10 in real time. The information processing apparatus 10 acquires the first biological information of the subject over time from the first measurement apparatus 11 and derives a timing suitable for measuring the second biological information of the subject based on the first biological information. The “timing suitable for measuring the second biological information” is a timing when the second biological information is estimated to be in the state suitable for diagnosis (that is, the second biological information of a desired result is obtained), and does not mean that the second biological information cannot be measured at other timings. In addition, the information processing apparatus 10 may instruct the second measurement apparatus 12 to measure the second biological information at the derived timing.

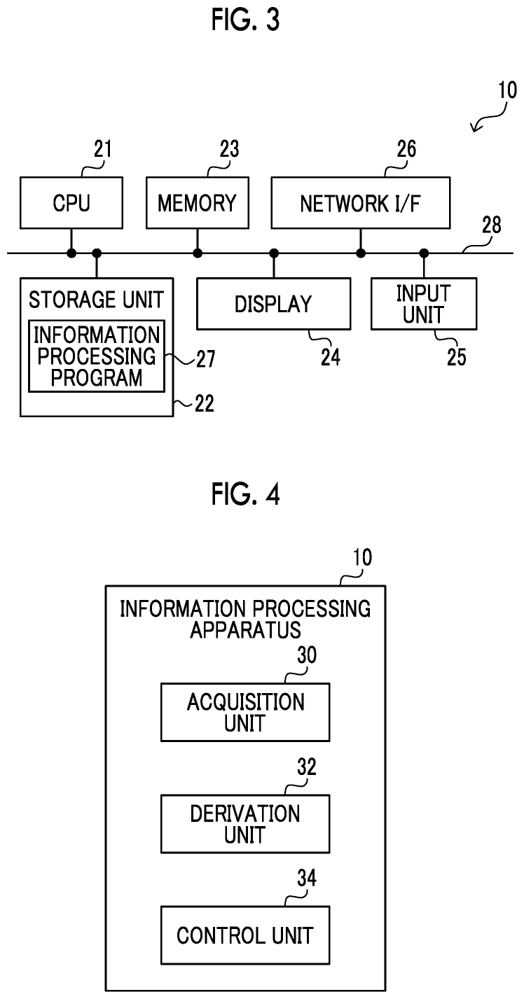

Hereinafter, a detailed configuration of the information processing apparatus 10 will be described. First, an example of a hardware configuration of the information processing apparatus 10 according to the present exemplary embodiment will be described with reference to FIG. 3. As illustrated in FIG. 3, the information processing apparatus 10 includes a central processing unit (CPU) 21, a non-volatile storage unit 22, and a memory 23 as a transitory storage region. In addition, the information processing apparatus 10 includes a display 24 such as a liquid crystal display, an input unit 25 such as a keyboard, a mouse, and a button, and a network interface (I/F) 26 that performs wired or wireless communication with the first measurement apparatus 11, the second measurement apparatus 12, and an external network (not illustrated). The CPU 21, the storage unit 22, the memory 23, the display 24, the input unit 25, and the network OF 26 are connected to be capable of exchanging various information with each other through a bus 28 such as a system bus and a control bus. For example, a personal computer, a server computer, a tablet terminal, a smartphone, and a wearable terminal can be applied as the information processing apparatus 10.

The storage unit 22 is implemented by a storage medium such as, for example, a hard disk drive (HDD), a solid state drive (SSD), and a flash memory. An information processing program 27 in the information processing apparatus 10 is stored in the storage unit 22. The CPU 21 reads out and loads the information processing program 27 from the storage unit 22 into the memory 23 and executes the loaded information processing program 27. The CPU 21 is an example of a processor according to the embodiment of the present disclosure.

Next, an example of a functional configuration of the information processing apparatus 10 according to the present exemplary embodiment will be described with reference to FIG. 4. As illustrated in FIG. 4, the information processing apparatus 10 includes an acquisition unit 30, a derivation unit 32, and a control unit 34. The CPU 21 functions as the acquisition unit 30, the derivation unit 32, and the control unit 34 by executing the information processing program 27.

The acquisition unit 30 acquires the first biological information of the subject over time from the first measurement apparatus 11. The derivation unit 32 derives the timing suitable for measuring the second biological information of the subject based on the first biological information acquired by the acquisition unit 30. The control unit 34 performs a control of presenting the timing derived by the derivation unit 32 and guidance corresponding to the timing using the display 24.

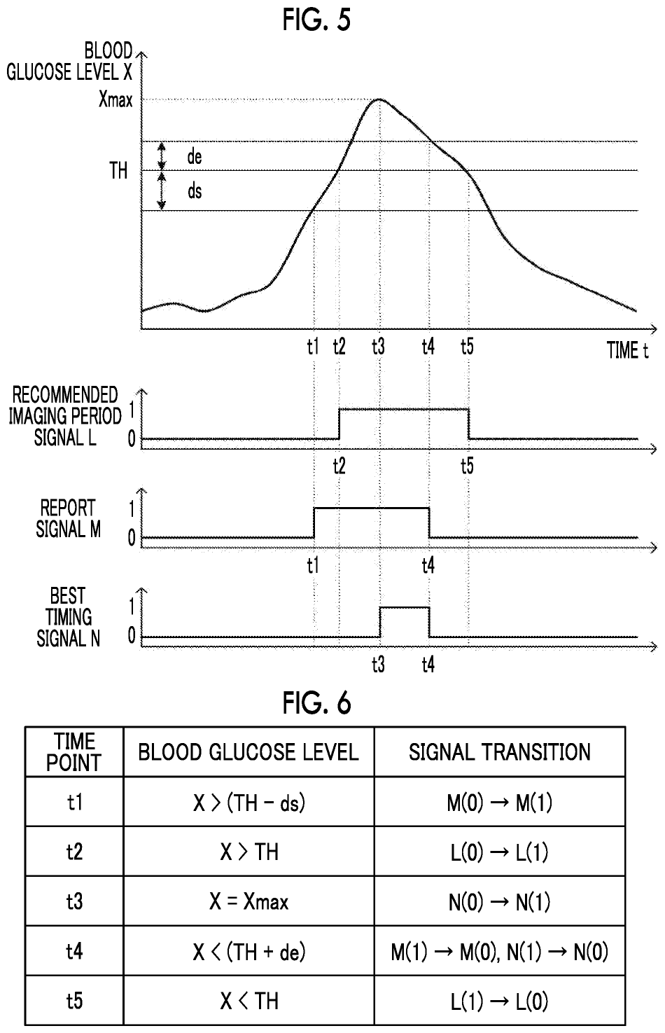

Hereinafter, an example of deriving a timing of capturing the fundus image as an example of the second biological information based on the blood glucose level as an example of the first biological information will be illustratively described. FIG. 5 illustrates an example of a change in a blood glucose level X after eating measured over time and an example of movement of each of signals L, M, and N that transitions between states of 0 and 1 in accordance with the blood glucose level X. The recommended imaging period signal L is a signal that has the state of 1 during a recommended imaging period suitable for capturing the fundus image. The report signal M is a signal for reporting the start and the end of the recommended imaging period by transitioning in state before the transition of the state of the recommended imaging period signal L. The best timing signal N is a signal that has the state of 1 at a time point that is most suitable for capturing the fundus image, which is when the blood glucose level X has a maximum value Xmax. Hereinafter, a state where the signal L is 0 will be denoted by L(0), and a state where the signal L is 1 will be denoted by L(1). The same applies to the signals M and N.

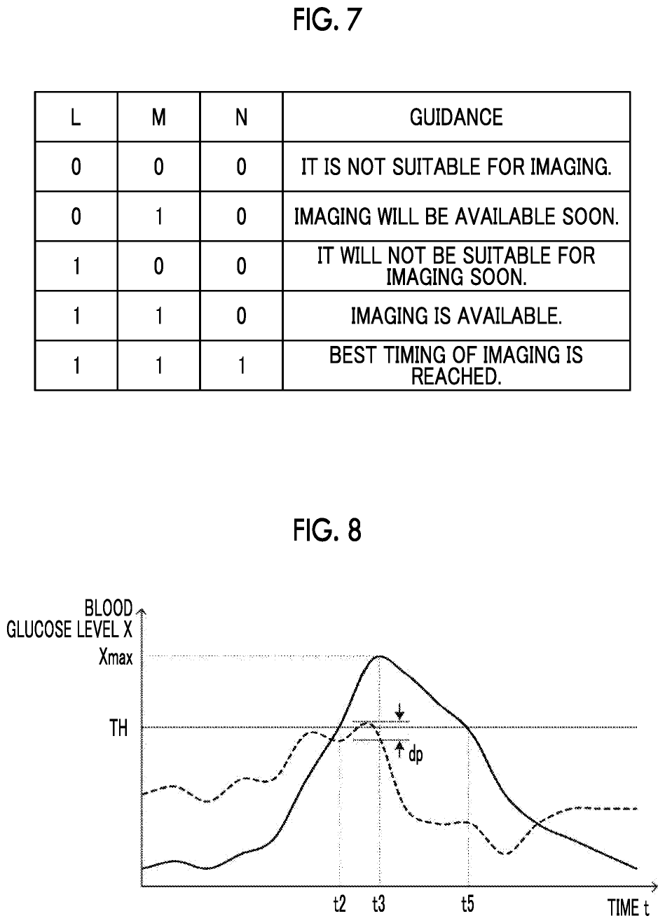

FIG. 6 is a table summarizing values of the blood glucose level at each of time points t1 to t5 in FIG. 5 and transitions of each of the signals L, M, and N. FIG. 7 illustrates an example of guidance presented by the control unit 34 in accordance with each of the signals L, M, and N using the display 24. In FIG. 7, combinations that each of the signals L, M, and N may not have (for example, a combination of L(0), M(0), and N(1)) are not described.

As illustrated in FIG. 5, the blood glucose level X is known to rapidly rise and rapidly fall after eating. The fundus image suitable for diagnosis of the diabetic retinopathy can be obtained by performing the fundus imaging at a timing near the blood glucose level X having the maximum value Xmax. The derivation unit 32 causes each of the signals L, M, and N to transition as illustrated in FIG. 5 and FIG. 6 based on the blood glucose level as the first biological information acquired by the acquisition unit 30.

For example, in a medical scene, preparation such as setting of the second measurement apparatus 12 and positioning of the subject may be required before capturing of the fundus image by the second measurement apparatus 12. In order to provide time for the preparation, the derivation unit 32 may also derive a timing for reporting the start of the recommended imaging period before a start timing of the recommended imaging period suitable for imaging (measuring) the fundus image (second biological information). Specifically, as illustrated in FIG. 5 and FIG. 6, the derivation unit 32 sets the report signal from M(0) to M(1) at the time point (a time point of X>(TH−ds)) t1 when the blood glucose level X has a value lower than a threshold value TH by a predetermined magnitude ds. The threshold value TH, for example, may be determined by a blood glucose level (140 mg/dL or the like) generally used in diagnosis of the diabetes or may be determined in accordance with a magnitude of rise from a fasting blood glucose level for each subject. At the time point t1, each signal has the states of L(0), M(1), and N(0). Thus, as illustrated in FIG. 7, the control unit 34 presents guidance “Imaging will be available soon.” that reports the start of the recommended imaging period of the fundus image.

In addition, for example, the derivation unit 32 derives the start timing of the recommended imaging period suitable for imaging (measuring) the fundus image (second biological information) based on the blood glucose level (first biological information). Specifically, as illustrated in FIG. 5 and FIG. 6, the derivation unit 32 sets the recommended imaging period signal from L(0) to L(1) at the time point (a time point of X>TH) t2 when the blood glucose level X has exceeded the predetermined threshold value TH. At the time point t2, each signal has the states of L(1), M(1), and N(0). Thus, as illustrated in FIG. 7, the control unit 34 presents guidance “Imaging is available.” that indicates being in the recommended imaging period of the fundus image.

In addition, for example, the derivation unit 32 may derive the most suitable timing for capturing (measuring) the fundus image (second biological information) from the subject based on the blood glucose level (first biological information). Specifically, as illustrated in FIG. 5 and FIG. 6, the derivation unit 32 sets the best timing signal from N(0) to N(1) at the time point t3 when the blood glucose level X has the maximum value Xmax. At the time point t3, each signal has the states of L(1), M(1), and N(1). Thus, as illustrated in FIG. 7, the control unit 34 presents guidance “Best timing of imaging is reached.” that indicates the most suitable timing for capturing the fundus image.

Derivation of the time point when the blood glucose level X has the maximum value Xmax can be performed based on, for example, a change in time of the blood glucose level (first biological information). FIG. 8 illustrates a time derivative of the blood glucose level X in FIG. 5 with a broken line. As illustrated in FIG. 8, it is perceived that the time derivative falls at the time point t3 when the blood glucose level X has the maximum value Xmax. Accordingly, the derivation unit 32 may derive the blood glucose level X as having the maximum value Xmax in a case where the time derivative of the blood glucose level X is decreased by a predetermined magnitude dp after the start of the recommended imaging period (that is, after the time point t2). While an example of deriving the time point when the blood glucose level X has the maximum value Xmax using the first order derivative of the blood glucose level X has been described in the example in FIG. 8, the present disclosure is not limited thereto. The time point when the blood glucose level X has the maximum value Xmax may be derived using a derivative of the second order to the fourth order or the like obtained by performing differentiation a plurality of times.

In addition, for example, it may be preferable to report the end of the recommended imaging period so that a measure such as preferentially imaging the subject provided with the report can be taken. Therefore, the derivation unit 32 may derive a timing for reporting the end of the period before an end timing of the recommended imaging period suitable for imaging (measuring) the fundus image (second biological information). Specifically, as illustrated in FIG. 5 and FIG. 6, the derivation unit 32 sets the report signal from M(1) to M(0) at the time point (a time point of X<(TH+de)) t4 when the blood glucose level X after having the maximum value Xmax (that is, after the time point t3) has a value higher than the threshold value TH by a predetermined magnitude de. In addition, at the time point t4, since it is considered that the best timing of imaging has ended, the best timing signal is set from N(1) to N(0). At the time point t4, each signal has the states of L(1), M(0), and N(0). Thus, as illustrated in FIG. 7, the control unit 34 presents guidance “It will not be suitable for imaging soon.” that reports the end of the recommended imaging period of the fundus image.

In addition, for example, the derivation unit 32 derives the end timing of the recommended imaging period suitable for imaging (measuring) the fundus image (second biological information) based on the blood glucose level (first biological information). Specifically, as illustrated in FIG. 5 and FIG. 6, the derivation unit 32 sets the recommended imaging period signal from L(1) to L(0) at the time point (a time point of X<TH) t5 when the blood glucose level X has lowered below the predetermined threshold value TH. At the time point t5, each signal has the states of L(0), M(0), and N(0). Thus, as illustrated in FIG. 7, the control unit 34 presents guidance “It is not suitable for imaging.” that indicates not being in the recommended imaging period of the fundus image.

Derivation of each timing by the derivation unit 32 is performed in real time in accordance with a change in the blood glucose level. On the other hand, in order for an imaging person to schedule imaging, it is preferable that the start timing and the end timing of the recommended imaging period of the subject and the best timing can be perceived in advance. Therefore, the derivation unit 32 may predict a change in time of the blood glucose level (first biological information) and derive each of the timings (that is, predict each of the timings) based on the predicted blood glucose level.

A method of predicting the change in time of the blood glucose level can employ a known method, as appropriate. For example, the derivation unit 32 may predict the change in time of the blood glucose level based on past data related to the blood glucose level of the subject. Specifically, for example, the change in time of the blood glucose level may be predicted using a representative value (for example, an average value and a median value) of the past data stored in advance in the storage unit 22. In addition, for example, the change in time of the blood glucose level may be predicted using a trained model that is trained to receive input of a trend of the blood glucose level up to the current time point and output a trend of the blood glucose level after the current time point.

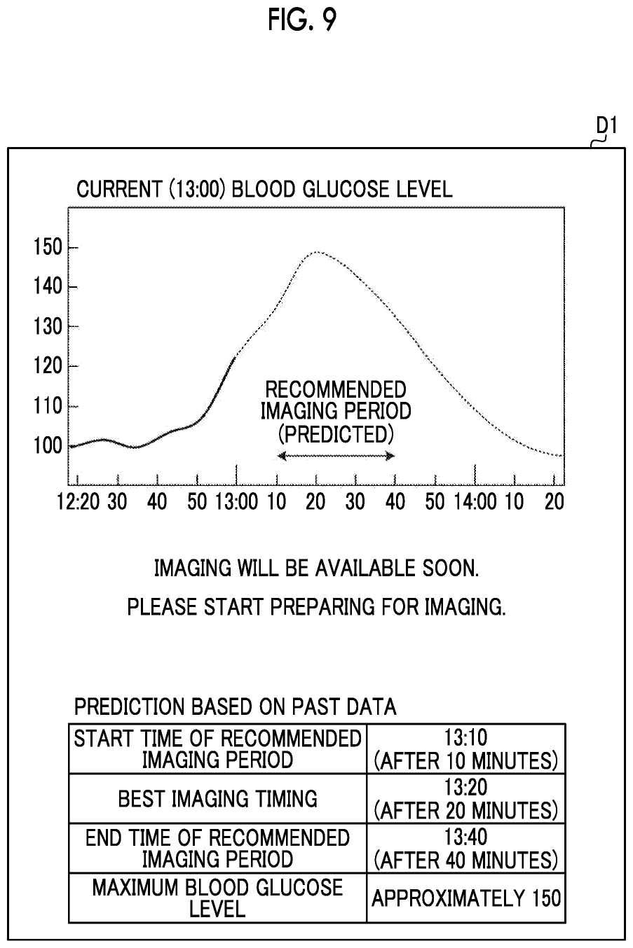

FIG. 9 illustrates an example of a screen D1 presented on the display 24 by the control unit 34. The screen D1 in FIG. 9 is presented at the time point t1 (that is, the timing for reporting the start of the period before the start timing of the recommended imaging period suitable for capturing the fundus image) in FIG. 5 and FIG. 6, and the time point t1 corresponds to 13:00. In FIG. 9, a record of the blood glucose level up to 13:00 is illustrated by a solid line, and prediction of the blood glucose level after 13:00 predicted by the derivation unit 32 is illustrated by a dotted line.

As illustrated in FIG. 9, the control unit 34 may perform a control of presenting the blood glucose level predicted by the derivation unit 32. In addition, the control unit 34 may perform a control of presenting the start timing and the end timing of the recommended imaging period and the best timing derived based on the blood glucose level predicted by the derivation unit 32. In addition, as illustrated in FIG. 9, the derivation unit 32 may predict the maximum blood glucose level, and the control unit 34 may perform a control of presenting the maximum blood glucose level predicted by the derivation unit 32.

In addition, the control unit 34 may instruct the second measurement apparatus 12, which measures the second biological information, to measure the second biological information at each of the timings derived by the derivation unit 32. Specifically, for example, in the examples in FIG. 5 and FIG. 6, the control unit 34 may instruct the second measurement apparatus 12 to capture the fundus image at t2 to t5 of the recommended imaging period derived by the derivation unit 32 as being suitable for imaging (measuring) the fundus image (second biological information). In addition, for example, the control unit 34 may instruct the second measurement apparatus 12 to capture the fundus image at the time point t3 derived by the derivation unit 32 as the most suitable timing for capturing (measuring) the fundus image (second biological information) from the subject.

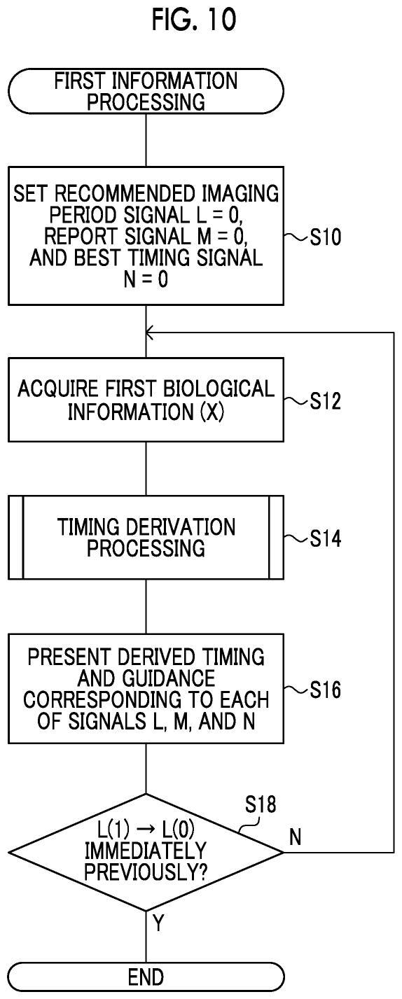

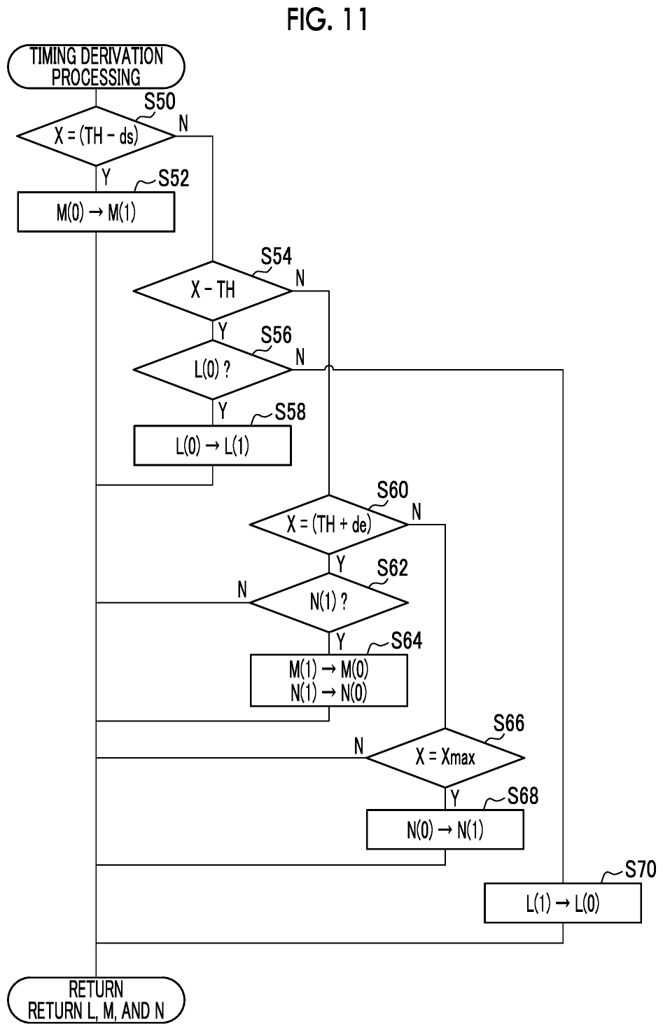

Next, action of the information processing apparatus 10 according to the present exemplary embodiment will be described with reference to FIG. 10 and FIG. 11. In the information processing apparatus 10, first information processing illustrated in FIG. 10 and timing derivation processing illustrated in FIG. 11 are executed by executing the information processing program 27 via the CPU 21. The first information processing is executed in a case where, for example, an instruction to start execution is provided by the user through the input unit 25.

In step S10, the derivation unit 32 sets each of the recommended imaging period signal L, the report signal M, and the best timing signal N to the state of “0”. In step S12, the acquisition unit 30 acquires the first biological information from the first measurement apparatus 11. Hereinafter, this first biological information (for example, the blood glucose level) will be denoted by X. In step S14, the derivation unit 32 executes the timing derivation processing illustrated in FIG. 11 based on the first biological information X acquired in step S12. In the timing derivation processing, the states of the recommended imaging period signal L, the report signal M, and the best timing signal N transition based on the first biological information X acquired in step S12. The state of each signal that has transitioned once is held until the state transitions again.

Here, the timing derivation processing executed in step S14 will be described with reference to FIG. 11. In step S50, the derivation unit 32 determines whether or not the first biological information X acquired in step S12 is a value lower than the predetermined threshold value TH by the predetermined magnitude ds (X=(TH−ds)). In a case where a positive determination is made in step S50 (that is, in the case of X=(TH−ds)), this means that the timing for reporting the start of the recommended imaging period (corresponding to the time point t1 in FIG. 5 and FIG. 6) is reached, and a transition is made to step S52. In step S52, the derivation unit 32 sets the report signal from M(0) to M(1) and returns each of the signals L, M, and N to the first information processing in FIG. 10.

On the other hand, in a case where a negative determination is made in step S50 (that is, in the case of X≠(TH−ds)), a transition is made to step S54. In step S54, the derivation unit 32 determines whether or not the first biological information X acquired in step S12 is equal to the predetermined threshold value TH (X=TH). In a case where a positive determination is made in step S54 (that is, in the case of X=TH), a transition is made to step S56, and the derivation unit 32 determines whether or not the current recommended imaging period signal L is in the state of “0”. In a case where a positive determination is made in step S56 (that is, in the case of L(0)), this means that the start timing of the recommended imaging period (corresponding to the time point t2 in FIG. 5 and FIG. 6) is reached, and a transition is made to step S58. In step S58, the derivation unit 32 sets the recommended imaging period signal from L(0) to L(1) and returns each of the signals L, M, and N to the first information processing in FIG. 10.

On the other hand, in a case where a negative determination is made in step S54 (that is, in the case of X≠TH), a transition is made to step S60. In step S60, the derivation unit 32 determines whether or not the first biological information X acquired in step S12 is a value higher than the predetermined threshold value TH by the predetermined magnitude de (X=(TH+de)). In a case where a positive determination is made in step S60 (that is, in the case of X=(TH+de)), a transition is made to step S62, and the derivation unit 32 determines whether or not the current best timing signal N is in the state of “1”. In a case where a positive determination is made in step S62 (that is, in the case of N(1)), this means that the timing for reporting the end of the recommended imaging period (corresponding to the time point t4 in FIG. 5 and FIG. 6) is reached, and a transition is made to step S64. In step S64, the derivation unit 32 sets the report signal from M(1) to M(0), sets the best timing signal from N(1) to N(0), and returns each of the signals L, M, and N to the first information processing in FIG. 10. On the other hand, in a case where a negative determination is made in step S62 (that is, in the case of N(0)), this means that the case corresponds to a time point between the time point t2 and the time point t3 in FIG. 5 and FIG. 6. Thus, each of the signals L, M, and N is returned to the first information processing in FIG. 10 without causing each signal to transition.

On the other hand, in a case where a negative determination is made in step S60 (that is, in the case of X≠(TH+de)), a transition is made to step S66. In step S66, the derivation unit 32 determines whether or not the first biological information X acquired in step S12 is equal to the maximum value Xmax (X=Xmax). In a case where a positive determination is made in step S66 (that is, in the case of X=Xmax), this means that the most suitable timing for capturing the fundus image (corresponding to the time point t3 in FIG. 5 and FIG. 6) is reached, and a transition is made to step S68. In step S68, the derivation unit 32 sets the best timing signal from N(0) to N(1) and returns each of the signals L, M, and N to the first information processing in FIG. 10.

On the other hand, in a case where a negative determination is made in step S56 (that is, in the case of L(1) at a time point of X=TH), this means that the end timing of the recommended imaging period (corresponding to the time point t5 in FIG. 5 and FIG. 6) is reached, and a transition is made to step S70. In step S70, the derivation unit 32 sets the recommended imaging period signal from L(1) to L(0) and returns each of the signals L, M, and N to the first information processing in FIG. 10. In addition, in a case where a negative determination is made in step S66 (that is, in the case of X≠Xmax), this means that the case does not correspond to any of each of the time points t1 to t5 when each signal transitions in FIG. 5 and FIG. 6. Thus, each of the signals L, M, and N is returned to the first information processing in FIG. 10 without causing each signal to transition.

In step S16 in FIG. 10, the control unit 34 performs a control of presenting the timing at the current time point and the guidance corresponding to the states of the recommended imaging period signal L, the report signal M, and the best timing signal N using the display 24. In step S18, the derivation unit 32 determines whether or not the recommended imaging period signal has transitioned from L(1) to L(0) in immediately previous step S14 (that is, whether or not the recommended imaging period signal is returned after step S70 is executed in the immediately previous timing derivation processing).

In a case where a negative determination is made in step S18 (that is, in a case where the recommended imaging period signal has not transitioned from L(1) to L(0) in immediately previous step S14), a return is made to step S12 while the state of each of the signals L, M, and N at the current time point is held. On the other hand, in a case where a positive determination is made in step S18 (that is, in a case where the recommended imaging period signal has transitioned from L(1) to L(0) in immediately previous step S14), this means that the current time point is the end timing of the recommended imaging period. Thus, the first information processing ends.

As described above, the information processing apparatus 10 comprises at least one processor, and the processor acquires the first biological information of the subject over time and derives the timing suitable for measuring the second biological information of the subject different from the first biological information based on the first biological information. That is, since the timing when the second biological information is in the state suitable for diagnosis can be derived, the second biological information for appropriate diagnosis can be measured.

### Second Exemplary Embodiment

In the first exemplary embodiment, the form of deriving the timing suitable for measuring the second biological information in real time based on the first biological information has been described. In an actual medical scene, a larger number of subjects than the number of second measurement apparatuses 12 may wait for measurement of the second biological information. In this case, it is desirable to provide support for scheduling which subject is measured at which time by deriving the timing suitable for measuring the second biological information in advance for each subject so that a medical worker can efficiently measure each subject.

Therefore, the information processing apparatus 10 according to the present exemplary embodiment has a function of scheduling a period for measuring the second biological information for each subject in addition to the function of the first exemplary embodiment. Hereinafter, an example of a functional configuration of the information processing apparatus according to the present exemplary embodiment will be described. Description of the same configuration as the first exemplary embodiment will be omitted in part.

The acquisition unit 30 acquires the first biological information over time related to each of a plurality of subjects. The derivation unit 32, for each subject, derives the timing (the start timing and the end timing of the recommended imaging period and the best imaging timing) suitable for measuring the second biological information different from the first biological information of the subject based on the first biological information. In this case, the derivation unit 32, for each subject, may predict the change in time of the first biological information and derive each of the timings based on the predicted first biological information. Each of the timings may be derived using the time point when the predicted first biological information is equal to the threshold value TH as the start timing and the end timing of the recommended imaging period and using the time point when the predicted first biological information has the maximum value as the best imaging timing (refer to FIG. 5).

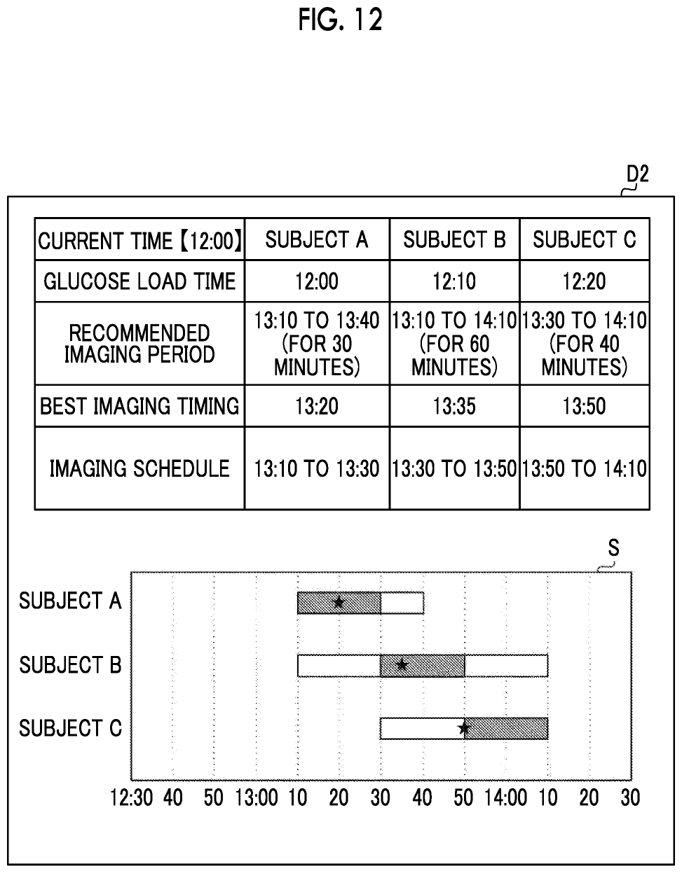

FIG. 12 illustrates an example of a screen D2 on which the recommended imaging period derived based on the predicted first biological information and the best imaging timing are presented by predicting the change in time of the first biological information for each of subjects A to C via the derivation unit 32. The “glucose load time” in FIG. 12 is a time when the subject takes glucose for intentionally creating a state of the high blood glucose spike after eating (that is, the state suitable for measuring the second biological information).

As illustrated in FIG. 12, a time taken from the glucose load time to the start timing of the recommended imaging period and a length and the like of the recommended imaging period vary for each individual. Therefore, it is preferable that the derivation unit 32 predicts the change in time of the first biological information based on the past data of each subject related to the first biological information. A specific method of deriving the start timing and the end timing of the recommended imaging period and the best imaging timing and a method of predicting the change in time of the first biological information via the derivation unit 32 are the same as those in the first exemplary embodiment. Thus, description thereof will be omitted.

In addition, the derivation unit 32 schedules the period for measuring the second biological information for each subject based on each of the derived timings. In a schedule S in FIG. 12, for each of the subjects A to C, the recommended imaging period of the fundus image (second biological information) is represented by a white frame, a scheduled period for capturing the fundus image in the recommended imaging period is filled with gray, and the predicted best imaging timing is represented by a star symbol. As illustrated in FIG. 12, the derivation unit 32 performs scheduling such that the periods (gray regions in FIG. 12) for measuring the second biological information for each of the subjects A to C do not overlap with each other in time.

Specifically, in a case where the recommended imaging periods derived for each of the subjects A to C overlap with each other, the derivation unit 32 schedules the period for measuring the second biological information by prioritizing the subject of which at least one of the start timing or the end timing of the recommended imaging period is earlier. For example, as illustrated in FIG. 12, the derivation unit 32 may schedule the period for measuring the second biological information by prioritizing the subject A of which the end timing of the recommended imaging period is earlier with respect to the subject A and the subject B of which the recommended imaging periods overlap with each other in part. In addition, for example, as illustrated in FIG. 12, the derivation unit 32 may schedule the period for measuring the second biological information by prioritizing the subject B of which the start timing of the recommended imaging period is earlier with respect to the subject B and the subject C of which the recommended imaging periods overlap with each other in part.

In addition, in a case where the recommended imaging periods derived for each of the subjects A to C overlap with each other, the derivation unit 32 may schedule the period for measuring the second biological information by placing the subject of which the period from the start timing to the end timing of the recommended imaging period is longer in the middle in order. For example, as illustrated in FIG. 12, the derivation unit 32 may schedule the period for measuring the second biological information by placing the subject B of which the period from the start timing to the end timing of the recommended imaging period is the longest in the middle in order. Doing so makes it easy to perform scheduling again (details will be described later).

In the schedule S in FIG. 12, the period for measuring the second biological information is scheduled for each subject based on each timing derived based on the change in time of the first biological information predicted by the derivation unit 32 for each subject. That is, since the schedule S in FIG. 12 is simply a schedule created based on the predicted change in time of the first biological information, the schedule S may not be consistent with the actual progress of the first biological information.

Therefore, the acquisition unit 30 may monitor the progress of the first biological information for each subject after the period for measuring the second biological information is scheduled by the derivation unit 32. In addition, the derivation unit 32 may schedule the period for measuring the second biological information again in a case where a difference between the progress of the first biological information monitored by the acquisition unit 30 and the predicted change in time of the first biological information falls outside an allowable range. Specifically, the derivation unit 32 may predict the change in time of the first biological information again based on the progress of the first biological information monitored by the acquisition unit 30 and derive each timing again based on the change in time of the first biological information predicted again. In addition, the derivation unit 32 may schedule the period for measuring the second biological information again based on each timing derived again.

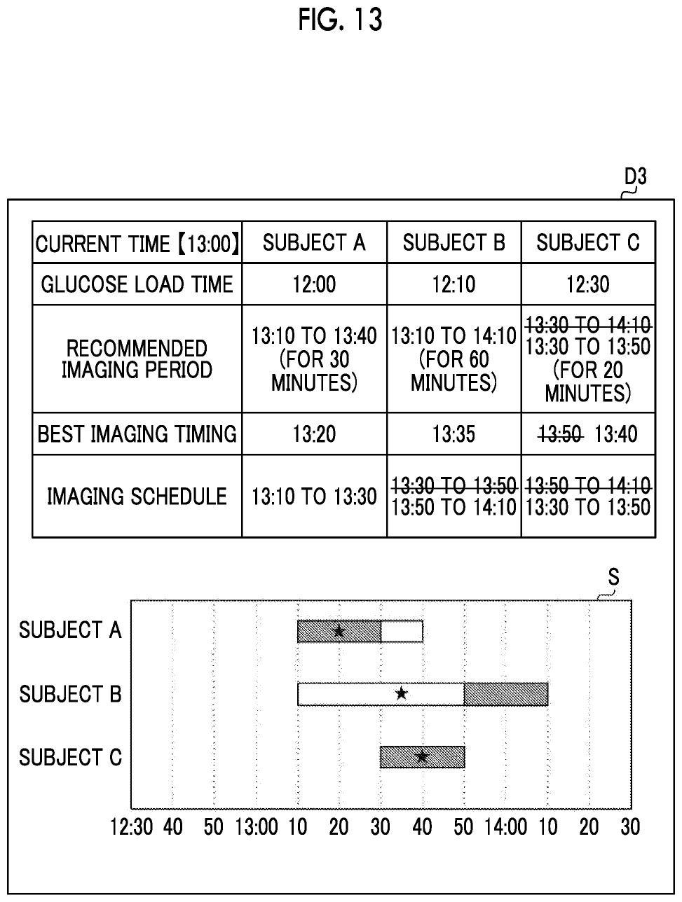

FIG. 13 illustrates an example of a screen D3 presented in a case where scheduling is performed again after one hour from FIG. 12. In FIG. 13, each timing after derivation performed again is described by crossing out each timing changed from the original prediction (the recommended imaging period and the best imaging timing in FIG. 12). In the example in FIG. 13, it is assumed that the difference between the progress of the first biological information monitored by the acquisition unit 30 and the predicted change in time of the first biological information falls outside the allowable range with respect to the subject C, and that the difference falls within the allowable range with respect to the subjects A and B.

As illustrated in FIG. 13, the derivation unit 32, with respect to the subject C, predicts the change in time of the first biological information again based on the progress of the first biological information monitored by the acquisition unit 30 and derives each timing again based on the change in time of the first biological information predicted again. Since the end timing of the recommended imaging period of the subject C is earlier than the subject B, the derivation unit 32 schedules the period for measuring the second biological information again to prioritize the subject C over the subject B.

In addition, the control unit 34 may instruct the second measurement apparatus 12, which measures the second biological information, to measure the second biological information during the period scheduled by the derivation unit 32. Specifically, for example, in the example in FIG. 13, the second measurement apparatus 12 may be instructed to measure the second biological information between 13:10 and 13:30 for the subject A, between 13:50 and 14:10 for the subject B, and between 13:30 and 13:50 for the subject C.

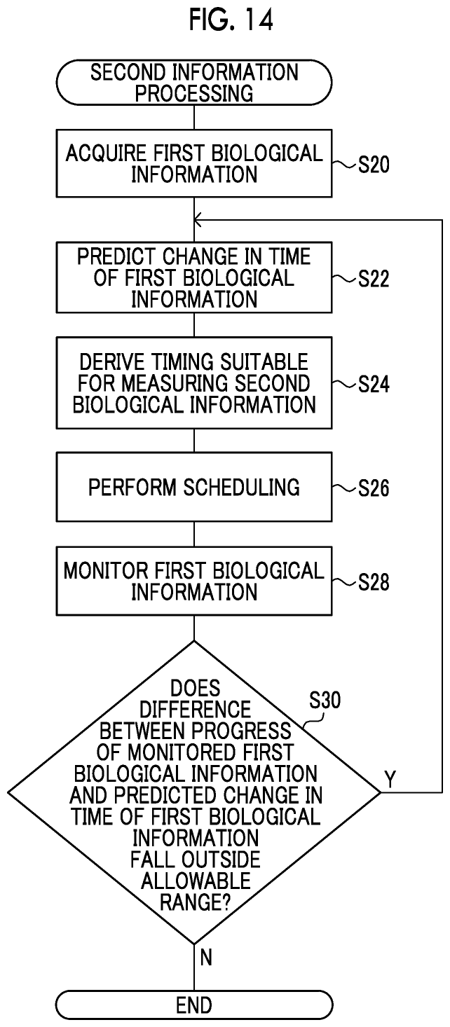

Next, action of the information processing apparatus 10 according to the present exemplary embodiment will be described with reference to FIG. 14. In the information processing apparatus 10, second information processing illustrated in FIG. 14 is executed by executing the information processing program 27 via the CPU 21. The second information processing is executed in a case where, for example, an instruction to start execution is provided by the user through the input unit 25.

In step S20, the acquisition unit 30 acquires the first biological information from the first measurement apparatus 11. In step S22, the derivation unit 32 predicts the change in time of the first biological information for each subject based on the first biological information acquired in step S20. In step S24, the derivation unit 32 derives the timing suitable for measuring the second biological information (for example, the start timing and the end timing of the recommended imaging period and the best imaging timing) based on the change in time of the first biological information predicted in step S22. In step S26, the derivation unit 32 schedules the period for measuring the second biological information for each subject based on each timing derived in step S24.

In step S28, the acquisition unit 30 monitors the progress of the first biological information of each subject. In step S30, the derivation unit 32, for each subject, determines whether or not the difference between the progress of the first biological information monitored in step S28 and the change in time of the first biological information predicted in step S22 falls outside the allowable range. In a case where a negative determination is made in step S30 (that is, in a case where the difference between the progress of the first biological information monitored in step S28 and the change in time of the first biological information predicted in step S22 falls outside the allowable range), the processing of steps S22 to S28 is performed again. On the other hand, in a case where a positive determination is made in step S30 (that is, in a case where the difference between the progress of the first biological information monitored in step S28 and the change in time of the first biological information predicted in step S22 falls within the allowable range), the second information processing ends.

As described above, the information processing apparatus 10 comprises at least one processor, and the processor acquires the first biological information over time related to each of the plurality of subjects, for each subject, derives the timing suitable for measuring the second biological information of the subject different from the first biological information based on the first biological information, and schedules the period for measuring the second biological information for each subject based on the derived timing. That is, since scheduling can be performed to measure the second biological information at the timing when the second biological information is in the state suitable for diagnosis, the second biological information for appropriate diagnosis can be measured.

The configuration of the information processing system 1 in each of the exemplary embodiments is not limited to the example illustrated in FIG. 1. For example, a part or all of the information processing apparatus 10, the first measurement apparatus 11, and the second measurement apparatus 12 included in the information processing system 1 may be an identical apparatus. For example, the information processing apparatus 10 may comprise the first measurement apparatus 11 that measures the first biological information, and the second measurement apparatus 12 that measures the second biological information.

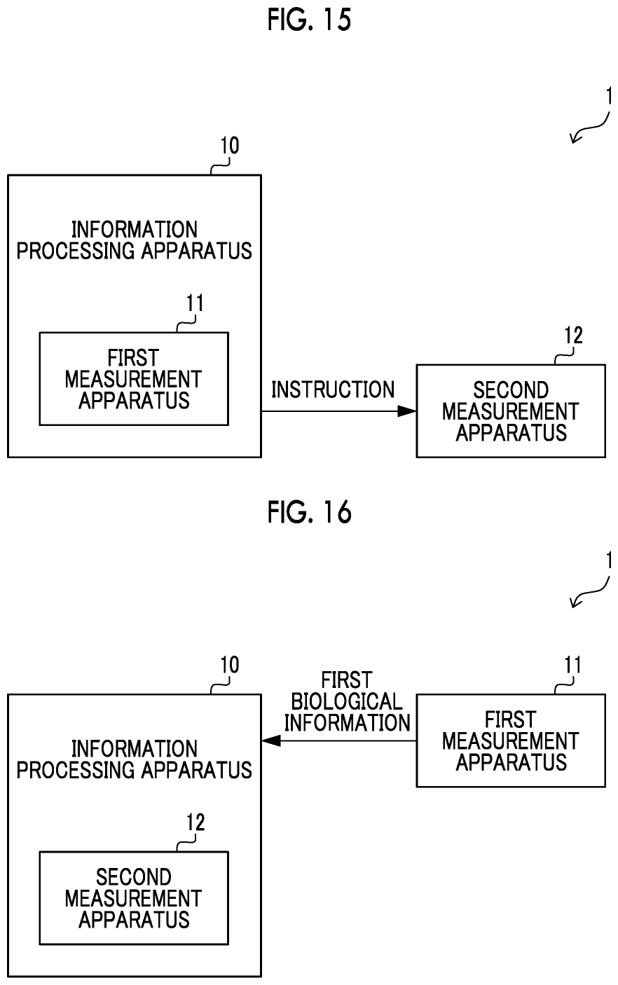

In addition, for example, as illustrated in FIG. 15, the information processing system 1 may be configured to comprise the information processing apparatus 10 encompassing the first measurement apparatus 11 that measures the first biological information, and the second measurement apparatus 12 that measures the second biological information in accordance with an instruction from the information processing apparatus 10. For example, a wearable terminal such as a smartwatch comprising a self-monitoring blood glucose meter and a sensor that measures the first biological information such as the heart rate and SpO2 may be applied as the information processing apparatus 10.

In addition, for example, as illustrated in FIG. 16, the information processing system 1 may be configured to comprise the information processing apparatus 10 encompassing the second measurement apparatus 12 that measures the second biological information, and the first measurement apparatus 11 that transmits the first biological information to the information processing apparatus 10. For example, a modality such as the medical image capturing apparatus may be applied as the information processing apparatus 10.

While one first measurement apparatus 11 and one second measurement apparatus 12 are illustrated in FIG. 1, FIG. 15, and FIG. 16, the present disclosure is not limited thereto. The information processing system 1 may comprise a plurality of the first measurement apparatuses 11 and a plurality of the second measurement apparatuses. In addition, each of the plurality of first measurement apparatuses 11 may measure the same type of the first biological information, or each may measure different types of the first biological information. Similarly, each of the plurality of second measurement apparatuses 12 may measure the same type of the second biological information, or each may measure different types of the second biological information.

In addition, in the exemplary embodiments, for example, the following various processors can be used as a hardware structure of a processing unit that executes various types of processing of the acquisition unit 30, the derivation unit 32, and the control unit 34. The various processors include, in addition to a CPU that is a general-purpose processor functioning as various processing units by executing software (program) as described above, a programmable logic device (PLD) such as a field programmable gate array (FPGA) that is a processor having a circuit configuration changeable after manufacture, a dedicated electric circuit such as an application specific integrated circuit (ASIC) that is a processor having a circuit configuration dedicatedly designed to execute specific processing, and the like.

One processing unit may be composed of one of the various processors or may be composed of a combination of two or more processors of the same type or different types (for example, a combination of a plurality of FPGAs or a combination of a CPU and an FPGA). In addition, a plurality of processing units may be composed of one processor.

A first example of a plurality of processing units composed of one processor is, as represented by computers such as a client and a server, a form of one processor composed of a combination of one or more CPUs and software, in which the processor functions as a plurality of processing units. A second example is, as represented by a system on chip (SoC) or the like, a form of using a processor that implements functions of the entire system including a plurality of processing units in one integrated circuit (IC) chip. In such a manner, various processing units are configured using one or more of the various processors as a hardware structure.

Furthermore, more specifically, an electric circuit (circuitry) in which circuit elements such as semiconductor elements are combined can be used as the hardware structure of the various processors.

While an aspect in which the information processing program 27 is stored (installed) in advance in the storage unit 22 has been described in the exemplary embodiments, the present disclosure is not limited thereto. The information processing program 27 may be provided in the form of a recording on a recording medium such as a compact disc read only memory (CD-ROM), a digital versatile disc read only memory (DVD-ROM), and a universal serial bus (USB) memory. In addition, the information processing program 27 may be provided in the form of a download from an external apparatus through a network. Furthermore, in addition to the information processing program, the disclosed technology is applied to a storage medium that stores the information processing program in a non-transitory manner.

In the disclosed technology, the exemplary embodiments can also be appropriately combined. Above described contents and illustrated contents are detailed descriptions for parts according to the embodiment of the disclosed technology and are merely an example of the disclosed technology. For example, description related to the above configurations, functions, actions, and effects is description related to an example of configurations, functions, actions, and effects of the parts according to the embodiment of the disclosed technology. Thus, unnecessary parts may be removed, new elements may be added, or parts may be replaced in the above described contents and in the illustrated contents without departing from the gist of the disclosed technology.

The disclosure of JP2021-069310A filed on Apr. 15, 2021 is incorporated in the present specification by reference in its entirety. All documents, patent applications, and technical standards disclosed in the present specification are incorporated in the present specification by reference to the same extent as in a case where each of the documents, patent applications, and technical standards are specifically and individually indicated to be incorporated by reference.

What is claimed is:

1. An information processing apparatus comprising at least one processor, wherein the processor is configured to:
acquire first biological information over time related to each of a plurality of subjects;
for each subject, derive a timing suitable for measuring second biological information of the subject different from the first biological information based on the first biological information; and
schedule a period for measuring the second biological information for each subject based on the derived timing.

2. The information processing apparatus according to claim 1, wherein the processor is configured to perform scheduling such that the periods for each subject do not overlap with each other.

3. The information processing apparatus according to claim 1, wherein the processor is configured to for each subject, derive the timing based on a change in time of the first biological information.

4. The information processing apparatus according to claim 1, wherein the processor is configured to for each subject, derive a start timing and an end timing of a period suitable for measuring the second biological information based on the first biological information.

5. The information processing apparatus according to claim 4, wherein the processor is configured to in a case where the timings derived for each subject overlaps with each other, schedule the period for measuring the second biological information by prioritizing a subject of which at least one of the start timing or the end timing is earlier.

6. The information processing apparatus according to claim 4, wherein the processor is configured to in a case where the timings derived for each subject overlaps with each other, schedule the period for measuring the second biological information by placing a subject of which a period from the start timing to the end timing is longer in a middle in order.

7. The information processing apparatus according to claim 1, wherein the processor is configured to for each subject, derive a most suitable timing for measuring the second biological information from the subject based on the first biological information.

8. The information processing apparatus according to claim 1, wherein the processor is configured to:
for each subject, predict a change in time of the first biological information; and
derive the timing based on the predicted first biological information.

9. The information processing apparatus according to claim 8, wherein the processor is configured to predict the change in time of the first biological information based on past data related to the first biological information for each subject.

10. The information processing apparatus according to claim 8, wherein the processor is configured to:
monitor progress of the first biological information for each subject after scheduling the period; and
in a case where a difference between the progress of the first biological information and the predicted change in time of the first biological information falls outside an allowable range, predict the change in time of the first biological information again, derive the timing again, and schedule the period again.

11. The information processing apparatus according to claim 1, wherein the processor is configured to present the scheduled period.

12. The information processing apparatus according to claim 1, wherein the processor is configured to instruct a second measurement apparatus that measures the second biological information to measure the second biological information in the scheduled period.

13. The information processing apparatus according to claim 1, wherein the first biological information aperiodically changes depending on behavior of the subject.

14. The information processing apparatus according to claim 1, wherein:
the first biological information indicates at least one of a body temperature, a heart rate, an electrocardiogram, an electromyogram, a blood pressure, an arterial oxygen saturation, a blood glucose level, or a lipid level, and
the second biological information indicates the electrocardiogram, an electroencephalogram, a medical image captured by a medical image capturing apparatus, and a result of at least one of a hematological test, an infectious disease test, a biochemical test, or a urine test.

15. The information processing apparatus according to claim 1, further comprising:
a first measurement apparatus that measures the first biological information; and
a second measurement apparatus that measures the second biological information.

16. An information processing system comprising:
the information processing apparatus according to claim 1;
a first measurement apparatus that measures the first biological information; and
a second measurement apparatus that measures the second biological information.

17. An information processing system comprising:
the information processing apparatus according to claim 1; and
a first measurement apparatus that measures the first biological information,
wherein the information processing apparatus further includes a second measurement apparatus that measures the second biological information.

18. An information processing system comprising:
the information processing apparatus according to claim 1; and
a second measurement apparatus that measures the second biological information,
wherein the information processing apparatus further includes a first measurement apparatus that measures the first biological information.

19. An information processing method comprising:
via a computer,
acquiring first biological information over time related to each of a plurality of subjects;
deriving, for each subject, a timing suitable for measuring second biological information of the subject different from the first biological information based on the first biological information; and
scheduling a period for measuring the second biological information for each subject based on the derived timing.

20. A non-transitory computer-readable storage medium storing an information processing program causing a computer to execute a process comprising:
acquiring first biological information over time related to each of a plurality of subjects;
deriving, for each subject, a timing suitable for measuring second biological information of the subject different from the first biological information based on the first biological information; and
scheduling a period for measuring the second biological information for each subject based on the derived timing.
